(19) United States
(12) Patent Application Publication
(10) Pub. No.: US 2022/0076828 A1
(43) Pub. Date: Mar. 10, 2022

(54) Context Aware Machine Learning Models for Prediction

(71) Applicant: Babylon Partners Limited, London (GB)

(72) Inventors: Yuanzhao ZHANG, London (GB); Robert WALECKI, London (GB); Iurii PEROV, London (GB); Adam Philip BAKER, George Town (KY); Christopher Robert HART, London (GB); Sara Marisa da Nazaré LOURENÇO, London (GB); Joanne Rebecca WINTER, London (GB); Saurabh JOHRI, London (GB)

(21) Appl. No.: 17/016,735

(22) Filed: Sep. 10, 2020

(51) Int. Cl. G16H 50/20; G06N 20/00; G16H 50/70; G16H 50/30; G06F 40/30

(52) U.S. Cl. CPC G16H 50/20; G06N 20/00; G06F 40/30; G16H 50/30; G16H 50/70

(57) ABSTRACT

A computer implemented method for developing a probabilistic graphical representation, the probabilistic graphical representation comprising nodes and links between the nodes indicating a relationship between the nodes, wherein the nodes represent conditions, the method comprising:
using a language model to produce a context aware embedding for said condition;
enhancing said embedding with one or more features to produce an enhanced embedded vector; and
using a machine learning model to map said enhanced embedded vector to a value, wherein said value is related to the node representing said condition or a neighbouring node,
wherein said machine learning model has been trained using said enhanced embedded vectors and observed values corresponding to said enhanced embedded vectors.

### FIELD

Embodiments described herein are related to methods of embedding text and methods of predicting results where there is insufficient data to make an accurate prediction, the predictions can be used to enhance a Graphical Representation.

### BACKGROUND

Obtaining accurate and comprehensive medical data is typically prohibitive. For instance, collecting data using epidemiological studies may take decades, and even then the values can be highly biased. Furthermore, emerging diseases and medical advances are two examples of circumstances whereby public health priorities shift rapidly and policy makers cannot wait for data to make thoroughly evidence-based decisions.

Accurate, comprehensive estimation of global health statistics is crucially important for informing health priorities and health policy decisions at global, national and local scales. Metrics such as the incidence and prevalence of different diseases need to be representative of the population of interest for them to be useful in tailoring health policies for different countries or different sub-populations within countries, More recently, comprehensive data on the burden of different diseases in many different settings has also become an important factor in the development of AI solutions addressing global healthcare needs.

Getting accurate estimates of the burden of different diseases globally is a challenging problem. Collecting high quality epidemiological data is not trivial; it takes a substantial amount of time, money and expertise to design rigorous data collection processes, to gather data, and to build infrastructure to support data collection on a routine or ad-hoc basis. This can be particularly problematic in developing countries where health systems are less robust and face difficulties such as lack of funding, staff shortages, and poor computer infrastructure. These problems can be compounded by the occurrence of natural disasters, disease epidemics, and civil unrest, which can disrupt existing healthcare systems.

Furthermore, graphical representations of data are used during automated diagnosis. Such models rely on the ability to understand and represent the relationship between conditions and symptoms. The data for these models and the construction of these models also requires considerable long term studies.

### BRIEF DESCRIPTION OF THE DRAWINGS

Embodiments will now be described with reference to the following figures in which:

FIG. 1 is a schematic of a medical diagnosis system;

FIG. 2 is a schematic of a simple PGM of the type that can be used in the inference engine of FIG. 1;

FIG. 3 is a flow diagram of a prediction method in accordance with an embodiment;

FIG. 4 is a flow diagram of a method of training a model to be used in the method of FIG. 3;

FIG. 5(a) and FIG. 5(b) are residual plots with residuals plotted in the y axis against the predicted value on the x-axis, in FIG. 5(a) residuals for a naïve baseline are plotted whereas in 5(b) residuals from a method in accordance with an embodiment are plotted for unseen countries;

FIG. 6(a) and FIG. 6(h) are residual plots, where FIG. 6(a) shows the predicted value with respect to GBD data and FIG. 6(b) shows the residual for the predicted value with respect to the data from peer-reviewed journals;

FIG. 7(a) and FIG. 7(b) are plots of MAE and concordance (respectively) for different age groups for previously unseen countries, previously unseen diseases and specific country disease pairs;

FIG. 8(a) and FIG. 8(b) are plots of MAE and concordance (respectively) for different disease categories;

FIGS. 9(a), 9(b), 9(c) and 9(d) are plots showing predictions of the incidence of subarachnoid haemorrhage, kidney cancer, liver cancer and psoriasis respectively, the darker line is the prediction from a model in accordance with an embodiment and the lighter line is the ground truth (GBD) data;

FIG. 10 is a flow diagram showing a method of producing an embedding in accordance with an embodiment;

FIG. 11 is a schematic showing concept embedding described with reference to FIG. 12;

FIG. 12 is a schematic showing descriptor embedding described with reference to FIG. 10;

FIG. 13 is a flow diagram showing a method for producing an augmented embedding for input into a neural network;

FIG. 14 is a schematic showing step S105 of FIG. 13;

FIG. 15 demonstrates a mapping from descriptors to contexts derived in step S105;

FIG. 16 demonstrates how the concept vectors are combined to produce a CCE;

FIG. 17 demonstrates how the CCE of FIG. 16 is augmented;

FIG. 18 is a schematic of a method in accordance with an embodiment for link prediction;

FIG. 19 is a schematic of a PGM for explaining link prediction;

FIG. 20 is a flow diagram showing an inference method using predictions produced by the method explained with reference to FIG. 3 and

FIG. 21 is a schematic of a system in accordance with an embodiment.

### DETAILED DESCRIPTION

In an embodiment, a computer implemented method for predicting a value of the prevalence or incidence of a condition in a population is provided, the method comprising:
using a language model to produce a context aware embedding for said condition;
enhancing said embedding with one or more labels to produce an enhanced embedded vector, said labels providing information concerning the population; and
using a machine learning model to map said enhanced embedded vector to said value,
wherein said machine learning model has been trained using said enhanced embedded vectors and observed values corresponding to said enhanced embedded vectors.

In a further embodiment, a computer implemented method for developing a probabilistic graphical representation is provided, the probabilistic graphical representation comprising nodes and links between the nodes indicating a relationship between the nodes, wherein the nodes represent conditions, the method comprising:
using a language model to produce a context aware embedding for said condition;
enhancing said embedding with one or more features to produce an enhanced embedded vector; and
using a machine learning model to map said enhanced embedded vector to a value, wherein said value is related to the node representing said condition or a neighbouring node,
wherein said machine learning model has been trained using said enhanced embedded vectors and observed values corresponding to said enhanced embedded vectors.

The disclosed system provides an improvement to computer functionality by allowing computer performance of a function not previously performed by a computer. Specifically, the disclosed system provides for the accurate prediction of a condition of a population from related data. The disclosed system solves this technical problem by providing the specific embedded representation of input text. This embedded representation draws on both linguistic considerations, but also the context of the text. This allows the embedded representation to be used to train a neural network where, for example, data from one country can be applied to another country and thus allows for the prediction of results where insufficient data is provided.

This, avoids the need to gather data for all possible conditions in all locations. It also means that the machine learning model used to predict the prevalence or incidence can be trained on less data allowing computational advantages and a reduced tine to train the model.

Many diagnostic systems, both medical and others, for example, fault diagnosis, use a probabilistic graphical representation (which can also be referred to as a probabilistic graphical model or “PGM”) which mathematically models the dependencies of the various conditions. The methods described herein allow for the enhancement of a PGM both in terms of the ability to add new nodes, since the data can be predicted for new nodes, e.g. the prevalence of a condition allow a node to be introduced for a condition, and the prediction of new links between conditions.

In an embodiment, the condition can be selected from a disease, symptom or risk factor

The labels can be one or more selected from location, age and sex of population. The labels can be encoded in a context aware manner such that similar labels are encoded with similar vectors. For example, for the label of location, locations with similar populations, GDP and climate can be encoded to have similar vectors. Other labels can be one hot encoded.

The above has discussed enhancing the embedded vector with an label, but it is also possible for the enhanced embedded vector to be produced from two conditions by:
using a language model to produce a context aware embedding for a further condition and concatenating the embedding for the further condition with that of the embedding for the said condition to produce an enhanced embedded vector,
wherein the value represents the probability that the two conditions occur together.

The above can be used to predict further links in the PGM by comparing the said value with a threshold, the method determining the presence of a link in the probabilistic graphical between the two conditions if the value is above the threshold and adding the link to the probabilistic graphical model.

In an embodiment, the language model is adapted to receive free text. In an embodiment, the language model is selected from BioBERT, Global Vectors for Word Representation (GloVe), the Universal Sentence Encoder (USE) or one of the GPT models. In further embodiments, a combination of language models are used. One or more of the language models may be been trained on a biomedical database.

As explained the above can be used to determine data for the incidence and prevalence of diseases where there is incomplete data. For example:

1. Where data is available on the incidence of the disease in other countries and data on the incidence of other diseases in the same country, but data is missing for a specific disease-country pairing.

2. Where data is available on the incidence of the disease in other countries, but is missing for the country of interest.

3. Where data is available on the incidence of other diseases in that same country, but no data is available on the disease of interest.

For the above, in an embodiment, the label is location and the method is used to determine a value for a condition in a specified location and the machine learning model has been trained on values for conditions including this condition for other locations and for other conditions for the specified location.

For the above, in a further embodiment, said label is location and the method is used to determine a value for a condition in a specified location and the machine learning model has been trained on values for conditions not including this condition for any location.

For the above, in a further embodiment, said label is location and the method is used to determine a value for a condition in a specified location and the machine learning model has been trained on values for conditions, but not on any values concerning the specified location.

It is possible for the enhanced embedded vector to comprise two embedded concepts and one or more labels.

In a further embodiment, a bespoke embedding can be used where using a language model to produce a context aware embedding for said condition comprises:
embedding input text into a first embedded space, wherein said first embedded space comprises first vector representations of descriptors of concepts in a knowledge base;
selecting the a nearest neighbours said first embedded space, wherein the nearest neighbours are first vector representations of descriptors and a is an integer of at least 2;
acquiring second concept vector representations of the concepts corresponding to the descriptors of said a nearest neighbours, wherein the second concept vector representations of said concepts is based on relations between said concepts; and
combining said second concept vector representations into a single vector to produce said context vector representation of said input text.

In an embodiment, combining said second concept vector representations comprises:
for each dimension of said concept vector representations, obtaining a statistical representation of the values for the same dimension across the selected concept vector representations to produce a dimension in said context vector representation of said input text. The statistical representations may be selected from: mean, standard deviation, min and max.

The above methods may be used within a diagnosis system to determine whether a user has a disease or some condition. Thus, in a further embodiment, a method of determining the likelihood of a user having a condition is provided, the method comprising:
inputting a symptom into an inference engine, wherein said inference engine is adapted to perform probabilistic inference over a probabilistic graphical model,
said probabilistic graphical model comprising diseases, symptoms and risk factors and the probabilities linking these diseases, symptoms and risk factors, and wherein at least one of the probabilities is determined using the method described above.

In a further embodiment, a computer-readable medium comprising instructions which, when executed by a computer, cause the computer to carry out the above method. The medium may a physical medium such as a flashdrive or a transitory medium for example a download signal.

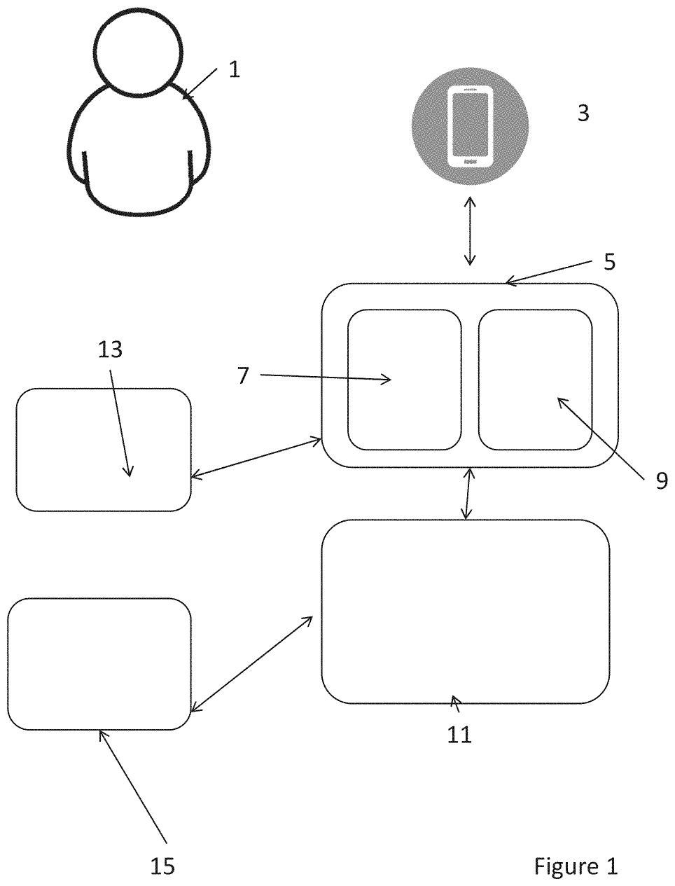

FIG. 1 is a schematic of a diagnostic system. In one embodiment, a user 1 communicates with the system via a mobile phone 3. However, any device could be used, which is capable of communicating information over a computer network, for example, a laptop, tablet computer, information point, fixed computer etc.

The mobile phone 3 will communicate with interface 5. Interface 5 has 2 primary functions, the first function 7 is to take the words uttered by the user and turn them into a form that can be understood by the inference engine 11. The second function 9 is to take the output of the inference engine 11 and to send this back to the user's mobile phone 3.

In some embodiments, Natural Language Processing (NLP) is used in the interface 5. NLP helps computers interpret, understand, and then use everyday human language and language patterns. It breaks both speech and text down into shorter components and interprets these more manageable blocks to understand what each individual component means and how it contributes to the overall meaning, linking the occurrence of medical terms to the Knowledge Graph. Through NLP it is possible to transcribe consultations, summarise clinical records and chat with users in a more natural, human way.

However, simply understanding how users express their symptoms and risk factors is not enough to identify and provide reasons about the underlying set of diseases. For this, the inference engine 11 is used. The inference engine is a powerful set of machine learning systems, capable of reasoning on a space of >100s of billions of combinations of symptoms, diseases and risk factors, per second, to suggest possible underlying conditions. The inference engine can provide reasoning efficiently, at scale, to bring healthcare to millions.

In an embodiment, the Knowledge Graph 13 is a large structured medical knowledge base. It captures human knowledge on modern medicine encoded for machines. This is used to allow the above components to speak to each other. The Knowledge Graph keeps track of the meaning behind medical terminology across different medical systems and different languages.

In an embodiment, the patient data is stored using a so-called user graph 15.

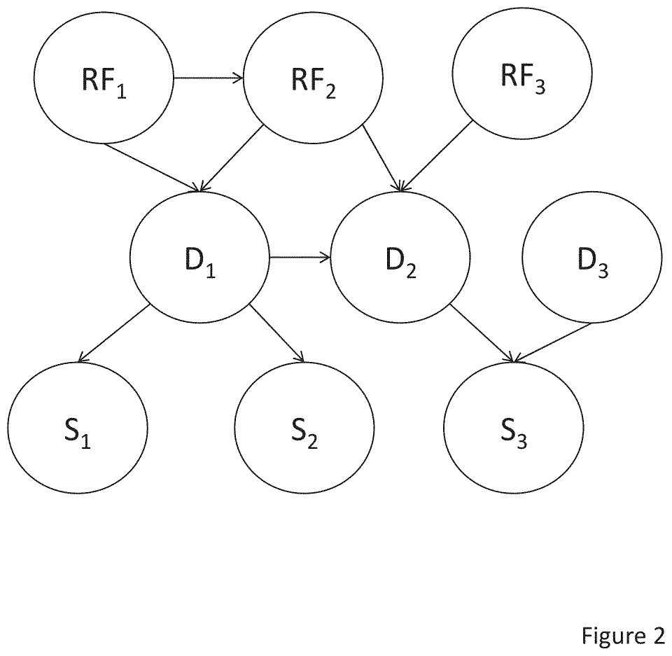

In an embodiment, the inference engine 11 comprises a generative model which may be a probabilistic graphical model or any type of probabilistic framework. FIG. 2 is a depiction of a probabilistic graphical model of the type that may be used in the inference engine 11 of FIG. 1.

In this specific embodiment, to aid understanding, a 3 layer Bayesian network will be described, where one layer related symptoms, another to diseases and a third layer to risk factors. However, the methods described herein can relate to any collection of variables where there are observed variables (evidence) and latent variables.

The graphical model provides a natural framework for expressing probabilistic relationships between random variables, to facilitate causal modelling and decision making. In the model of FIG. 2, when applied to diagnosis, D stands for disease, S for symptom and RF for Risk Factor. Three layers: risk factors, diseases and symptoms. Risk factors causes (with some probability) influence other risk factors and diseases, diseases causes (again, with some probability) other diseases and symptoms. There are prior probabilities and conditional marginals that describe the “strength” (probability) of connections.

In this simplified specific example, the model is used in the field of diagnosis. In the first layer, there are three nodes S₁, S₂ and S₃, in the second layer there are three nodes D₁, D₂ and D₃ and in the third layer, there are three nodes RF₁, RF₂ and RF₃.

In the graphical model of FIG. 2, each arrow indicates a dependency. For example, D₁ depends on RF₁ and RF₂. D₂ depends on RF₂, RF₃ and D₁. Further relationships are possible. In the graphical model shown, each node is only dependent on a node or nodes from a different layer. However, nodes may be dependent on other nodes within the same layer.

The embodiments described herein relate to the inference engine.

In an embodiment, in use, a user 1 may inputs their symptoms via interface 5. The user may also input their risk factors, for example, whether they are a smoker, their weight etc. The interface may be adapted to ask the patient 1 specific questions. Alternately, the patient may just simply enter free text. The patient's risk factors may be derived from the patient's records held in a user graph 15. Therefore, once the patient identified themselves, data about the patient could be accessed via the system.

In further embodiments, follow-up questions may be asked by the interface 5. How this is achieved will be explained later. First, it will be assumed that the patient provide all possible information (evidence) to the system at the start of the process.

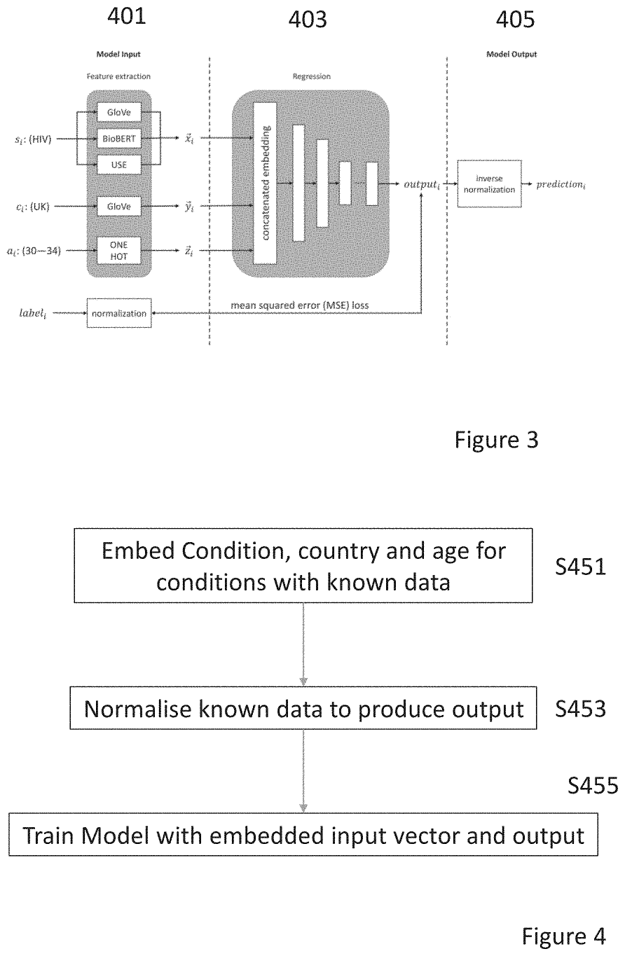

The evidence will be taken to be the presence or absence of all known symptoms and risk factors. For symptoms and risk factors where the patient has been unable to provide a response, these will assume to be unknown. However, some statistics will be assumed, for example, the incidence and/or prevalence of a disease in the country relevant to the user. This data is sometimes hard to obtain, the methods that will be described with reference to FIGS. 3 to 16 will show how to obtain a prediction of these figures.

When performing approximate inference, the inference engine 11 requires an approximation of the probability distributions within the PGM to act as proposals for the sampling.

In a very simple example, looking at FIG. 2, if a user of the system has a symptom S₃, their likelihood of having disease D₃ will be P(D₃|S₃) which can be written as:

$$P ( {D}_{3} \vert {S}_{3} ) = \frac{P ( {S}_{3} \vert {D}_{3} ) P ( {D}_{3} )}{P ( {S}_{3} )} \qquad Equation 1$$

To determine the above, one of the unknown quantities is P(D₃) which is the incidence of disease D₃. This can be determined using the methods taught in the following embodiments. Typically, P(D₃) will be location dependent and possible gender and age dependent Using the following embodiments P(D₃) may be estimated even if D₃ is an unknown new condition or if there is no data available for the country of interest.

Due to the size of the PGM, it is not possible to perform exact inference in a realistic timescale. Therefore, the inference engine 11 performs approximate inference.

These types of networks usually have three layers of nodes: the first level contains binary nodes that are risk factors; the second level, diseases; and the last level, symptoms. The network structure is designed by medical experts who assess whether there exists a direct relationship or not between a given pair of nodes.

However, there is a need to be able to obtain data for such networks. Obtaining accurate and comprehensive medical data is typically prohibitive. For instance, collecting data using epidemiological studies is costly and may take decades to complete.

Embodiments described herein allow inference to be provided for situations where the underlying data required to provide the probability distributions in the PGM is not available.

FIG. 3 is a diagram of a system in accordance with an embodiment.

There is already data available concerning the incidence and prevalence of diseases. For example, “The Global Burden of Disease (GBD)” study, conducted by the Institute for Health Metrics and Evaluation (IHME), aims to systematically and scientifically quantify health losses globally. The GBD captures data from 195 countries globally, and combines these data to produce accurate age- and sex-specific estimates of the incidence, prevalence, and rates of disability and mortality that are caused by over 350 diseases and injuries. Also, data is available from many sources, including surveys, administrative data (including vital registration data, census data, epidemiological and/or demographic surveillance data), hospital data, insurance claims data, disease registries and other related sources such as that published in the scientific literature. These data sources can be used as ground truths for training the models that will be explained below.

In the following, the disease incidence estimates produced by the deep learning models were validated using data published in the scientific literature and in national reports.

The system of FIG. 3 comprises 3 stages: an input stage 401, a regression stage 403 and an output stage 405. FIG. 3 is an illustration of machine learning pipeline we used for estimation of disease incidence. sᵢ represents the sentence embeddings of the disease of interest (e.g. HIV), cᵢ represents the embedding of the country of interest (e.g. UK), aᵢ represents the age group of interest (e.g. 30-34 years), and labelᵢ represents the ground-truth value (from the GBD study).

In the input stage 401, and input is formed which, in this example, comprises 3 sections each section is represented by a vector:

Section 1—Feature extraction from condition sᵢ represented as \vec{x}ᵢ,

Section 2—Feature extraction from country cᵢ represented as \vec{y}ᵢ,

Section 3—Feature extraction from age aᵢ represented as \vec{z}ᵢ

The three vectors \vec{x}ᵢ, \vec{y}ᵢ and \vec{z}ᵢ are concatenated together to provide a concatenated embedding to regression stage 403.

The feature extraction to produce \vec{x}ᵢ will now be explained. There are many methods for learning word embeddings from text. Words are generally represented as binary, one-hot encodings which map each word in a vocabulary to a unique index in a vector. These word encodings can then be used as inputs to a machine learning model, such as a neural network, to learn the context of words. The information encoded in these embeddings is tied to the text that was used to train the neural network. Word embeddings can discover hidden semantic relationships between words and can compute complex similarity measures. If these embeddings were obtained from training on different data sources, the context encoded would likely differ, Consequently, better performance in downstream tasks will be linked to the information content encoded in these dense representations of words and its relationship with the task itself.

In the following embodiment, different types of word representations are discussed, obtained by different modeling strategies, on the downstream task of predicting disease incidence. This is performed by using the embeddings as inputs to a neural network for estimating disease incidence. The word embedding methods that are used are detailed below,

### Global Vectors for Word Representation

The Global Vectors for Word Representation (GloVe) model is built on the word2vec method which initially converts words to numeric values. The GloVe model then learns its embeddings from a co-occurrence matrix of words, where each potential combination of words is represented as an entry in the matrix as the number of times the two words occur together within a pre-specified context window. This window moves across the entire corpus. In this work, we used the pre-trained GloVe model trained on common crawl data from raw web page data.

### BioBERT

Bidirectional Encoder Representations from Transformers (BERT) is a contextualized word representation model which learns the context for a given word from the words that precede and follow it in a body of text. In the following example BioBERT is used which is a model initialized with the general BERT model but pre-trained on large-scale biomedical corpora such as PubMed abstracts and PMC full-text articles. This enables the model to learn the biomedical context of words.

### Universal Sentence Encoder

The Universal Sentence Encoder (USE) is a language model which encodes context-aware representations of English sentences as fixed-dimension embeddings.

In addition to using each of the above language models individually, feature fusion is used to combine the three word embeddings into a single vector by concatenation. The neural network was then trained on the combined representation as shown in FIG. 3.

The process for training a neural network to predict disease incidence rates is illustrated in FIG. 3. The neural network consists of several hidden layers, which perform a non-linear transformation of the input features. In an embodiment, the neural network is a multi-layer-perceptron with 5-layer funnel architecture, where the first layer has 256 nodes, 2nd layer has 128, 3rd layer has 64, 4th layer has 32 fifth layer has 16 and the output is a single node. These input features consist of embeddings of disease, country, and age group. The neural network outputs a prediction for the incidence of a specified disease. Prior to training, the values for disease incidence are pre-processed with a log transformation. An inverse log transformation must therefore be applied to the neural network output to obtain the disease incidence rate.

FIG. 4 is a simple flow diagram showing the training of the model described with reference to FIG. 3.

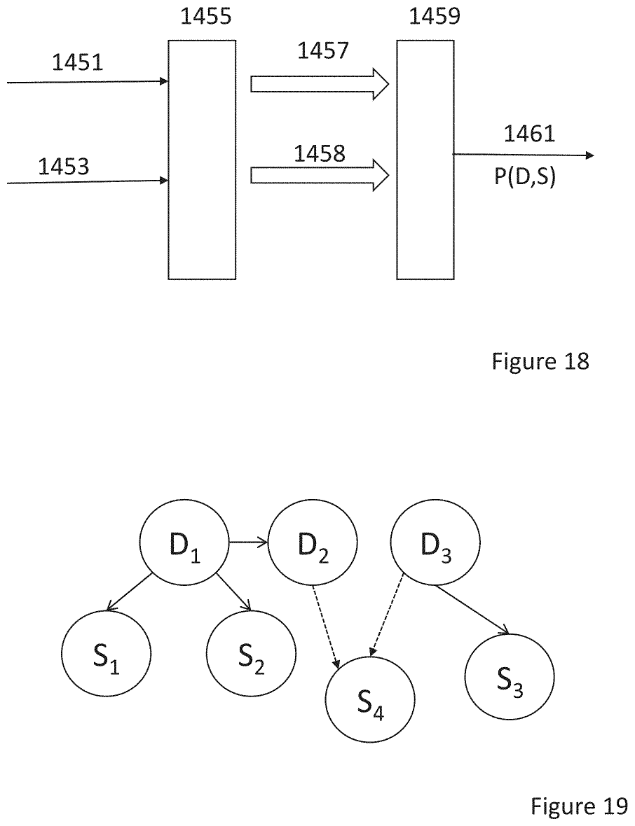

In step S451, pairs of embedded vectors and prevalence/incidence values as required are obtained. If the model is to be trained for incidents prediction, then incidence values are used, if the model is to be trained for prevalence prediction then prevalence values used. The embedded vectors are constructed as explained above in relation to FIGS. 3 and 401. The values are selected from GBD or some other established source. For ease of explanation, in this example, it will be assumed that incidence values are being predicted. However, prevalence values be predicted in exactly the same manner. It is also possible to predict conditional probabilities. For this, the enhanced embedded vector can comprise two conditions and the probability of one condition being present given the presence of the other can be determined. This will be explained in more detail with reference to FIGS. 18 and 19.

As noted above, the embedding used to produce the input vector can handle free speech. Therefore, it is possible to use training data that is derived from free text scientific papers and text books, reports etc.

As explained above, the incidence values are then normalised in step S453 In an example, this can be done by pre-processing with a log 10 transformation and then normalising. These normalised values then used to train the model in step S455. Any training method can be used, for example forward and back propagation.

In a first example, the system of FIG. 3 is used for specific disease-country pairs. This task simulates a scenario where it is needed to predict incidence rates for specific diseases in a selected set of countries. This is important if data points are missing or are difficult to collect in the target country. For this application, we have data of the target disease in other countries, and data of other diseases in the target country yet data for a specific target disease-country pair is missing.

In a second example, incidence values for previously unseen countries are predicted. There may be cases where there is no high-quality data available in countries with poor healthcare and data infrastructure. For these situations, it may be desirable to predict incidence rates of all diseases. For this application the case is simulated where there is no data for any disease in the target country but complete incidence data for all others.

In a third example, previously unseen diseases are predicted. This represents a situation where there is a key disease for which incidence data is difficult to obtain. This application consequently deals with the prediction of disease incidence rates for a given disease. In this case, incidence data is available for other diseases, but there is no data about the new, ‘unseen’ disease in any country.

Using the above, the condition is a disease and disease/condition embeddings are produced using each of the methods described above. For feature extraction for the country, in this example, GloVe is used to create representations of countries.

In this example, 20 age groups of 5-year periods (0-4, 5-9, . . . , 95+) were represented as binary one-hot vectors. Representing age groups in this way means that they are treated as separate categories, so that non-linear associations between incidence and age can easily be modelled.

The results that are presented below were modelled using standard 10-fold cross validation; the model parameters were estimated using 90% of the data, and validated on the remaining 10%. This avoids over-optimistic estimates of the model's performance, which can arise if the model is trained and tested on the same data. This process was repeated ten times with different, discrete 90/10 splits of the data.

The model shown in FIG. 3 was trained using estimates of the incidence of 199 diseases, across 195 countries and 20 age groups, sourced from the GBD study. A subset of data points with 0 incidence values was removed from the original GBD study (132,903/626,580 data points, 21%). Zero incidence can happen either because data is not available or the actual incidence value is zero for some specific data entries. Since the distribution of disease incidence values was highly skewed, in this example, the data was log-transformed to base 10. The predictions from the model were inverse log-transformed to derive estimates of disease incidence.

For each of the three different examples outlined above, cross-validation was performed as follows:

For Example 1, each fold contains randomly selected country-disease pairs, where it is possible that data from the same disease or country can occur in the training and validation set but not both. This model is optimized for predicting disease incidence for country-disease combinations the model has not seen before, e.g. HIV in Singapore. In this example, the training data may contain disease incidence estimates for other diseases in Singapore, and for HIV in other countries. The model is therefore able to learn from these combinations of samples and then to predict the incidence for different disease country pair.

For Example 2, it was ensured cross-validation was independent of the country. Within each fold of the data, the model was trained on data from 90% of countries, and validated on data from the remaining 10% of countries.

For Example 3, it was ensured that cross-validation was independent of disease, but not country. Within each data fold, the model was trained on data from 90% of diseases, and validated on data using the remaining 10% of diseases.

For each of the above Examples 1 to 3, the performance of the neural network in estimating disease incidence, using each of the language models discussed above was compared. For each prediction, the predictive power of the embeddings was compared to two separate baselines. The result was first compared them to the global average for the disease and/or country of interest. Secondly, the estimates were compared with the incidence values reported in the GBD study.

The mean absolute error (MAE) in log 10 space was used to evaluate the performance of the disease incidence estimation. For example, a prediction with MAE of 0.2 is either 1.58 times larger or lower than the “ground truth” value. The factor of 1.58 is computed by inverse transformation (10^{0.2}=1.58). To measure the similarity of relative rankings of the estimates (in the cases discussed here, between the predictions and the disease name labels in the GBD study), the inter-group concordance ρ_{c} ranking was calculated whose values are bounded between 0 (worst) and 1 (best).

The performance of each language embedding was evaluated based on the three possible applications and report results for both the GBD, cross-validation results and the independent test set.

Results for the performance across various embeddings are reported for the GBD data and independent test data in Tables 1, 2, and 3. On average, models that exploited BioBERT embeddings saw the best performance. This is exemplified in all applications across both validation datasets where the BioBERT model saw consistently low MAE and high concordance scores.

Whilst most embedding methods produced accurate incidence estimates in the GBD dataset, it is apparent that BioBERT, followed by GloVe embeddings produced the best results in the independent test set when compared to USE, For instance, BioBERT and GloVe had an MAE of 0.157 and 0.157 with concordance of 0.990 and 0.988 respectively compared to an MAE of 0.168 and a concordance of 0.985 for USE in the specific disease-country pairs application (Table 1). This illustrates that these embeddings contain informative, contextual information. This is validated in the Binary model, which used one-hot encoded representations and suffered in performance as seen in the previously unseen diseases (Table 2) and previously unseen countries (Table 3) and applications.

A minor ablation study was performed on the BioBERT embeddings by comparing the performance of the neural network method (BioBERT) with a ridge regression that used BioBERT features (BioBERT*). The neural network method saw consistently better results compared to the ridge regression across all applications in the GBD datasets.

The performance of most models was consistently high in the specific disease-country pairs application (Table 1) and previously unseen countries (Table 3). However, there was a marked decrease in the validation metrics within the previously unseen diseases application (Table 2), For instance, the MAE of BioBERT rose from 0.157 (Table 1) and 0.197 (Table 3) to 0.781 whilst the concordance (ρ_{c}) of GloVe for instance dropped from 0.988 (Table 1) and 0.955 (Table 3) to 0.775.

TABLE 1
Model performance (mean absolute error (MAE) and concordance (ρ_{c})) with different input features
for specific disease-country pairs on GBD data. The data is shown for Global which just returns
the average incidence value across all IHME countries. This is used as a baseline and the average
incidence of a disease is compared with the true values of all countries. RidgeReg also serves as
baseline where BioBERT feature + ridge regression model is used, OneHot^{d}, OneHot^{c}, OneHot^{d, c}
the NN models trained with binary representations which used one-hot encoded representations of
(d) disease, (c) country, and (d,c) for both, disease and country. (Example 1)

| Data | | | | | | | | | |
|---|---|---|---|---|---|---|---|---|---|
| Model | Global | RidgeReg | OneHot^{d} | OneHot^{c} | OneHot^{d, c} | BioBERT | GloVe | USE | Fusion |
| | Training and Validation (from the GBD study) | | | | | | | | |
| MAE. | .207 | .559 | .166 | .155 | .152 | .157 | .157 | .168 | .157 |
| ρ_{c} | .952 | .867 | .987 | .988 | .990 | .990 | .988 | .985 | .988 |
| | Test (from the epidemiological literature) | | | | | | | | |
| MAE. | N/A | 1.13 | N/A | N/A | N/A | .835 | .910 | 1.06 | 3.78 |
| ρ_{c} | N/A | 0.97 | N/A | N/A | N/A | .977 | .970 | .960 | .938 |

TABLE 2
Model performance(mean absolute error and concordance) with different
input features for previously unseen diseases on GBD data. The explanation
of the data labels is given above for table 1. (Example 2)

| Data | | | | | | | | | |
|---|---|---|---|---|---|---|---|---|---|
| Model | Global | RidgeReg | OneHot^{d} | OneHot^{c} | OneHot^{d, c} | BioBERT | GloVe | USE | Fusion |
| | Training and Validation (from the GBD study) | | | | | | | | |
| MAE. | N/A | 1.03 | N/A | .805 | N/A | .781 | .807 | .765. | .736 |
| ρ_{c} | N/A | .726 | N/A | .790 | N/A | .796 | .775 | .826 | .806 |
| | Test (from the epidemiological literature) | | | | | | | | |
| MAE. | N/A | 1.02 | N/A | N/A | N/A | .933 | .989 | 1.01 | 2.43 |
| ρ_{c} | N/A | .982 | N/A | N/A | N/A | .967 | .971 | .962 | .931 |

TABLE 3
Model performance(mean absolute error and concordance) with different
input features for previously unseen countries on GBD data. The explanation
of the data labels is given above for table 1. (Example 3)

| Data | | | | | | | | | |
|---|---|---|---|---|---|---|---|---|---|
| Model | Global | RidgeReg | OneHot^{d} | OneHot^{c} | OneHot^{d, c} | BioBERT | GloVe | USE | Fusion |
| | Training and Validation (from the GBD study) | | | | | | | | |
| MAE. | .212 | .562. | .204 | N/A | N/A | .197 | .198 | .209 | .196. |
| ρ_{c} | .965 | .866 | .953 | N/A | N/A | .954 | .955 | .953 | .955 |
| | Test (from the epidemiological literature) | | | | | | | | |
| MAE. | N/A | 1.12. | N/A | N/A | N/A | .881 | .921 | 1.07 | .937 |
| ρ_{c} | N/A | .976 | N/A | N/A | N/A | .972 | .977 | .970 | .978 |

The performance of the network trained with BioBERT features was examined across diseases with different magnitudes of incidence rate.

The distribution of errors was compared with the baseline model that predicted incidence rates using a global average estimate.

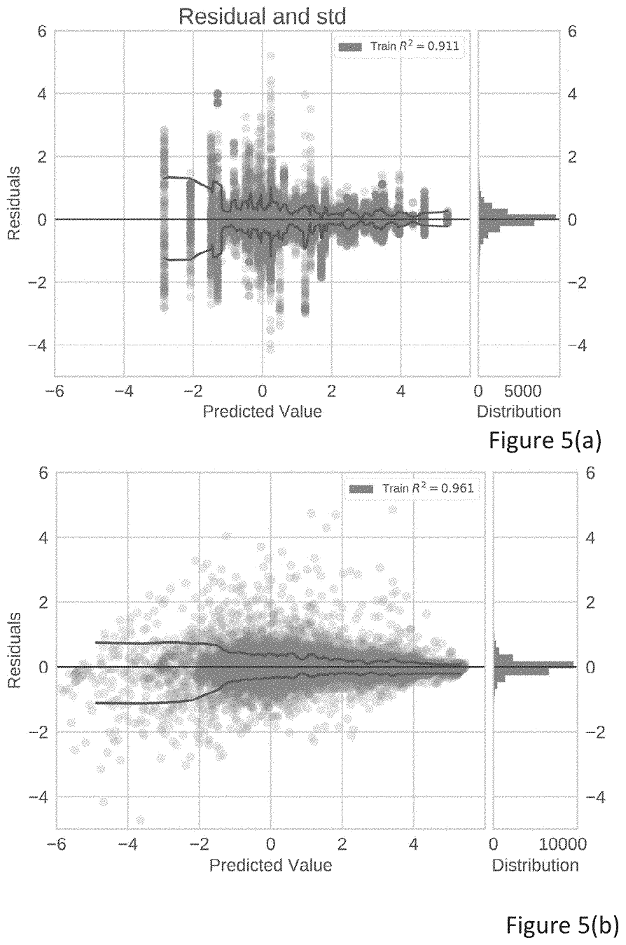

This is shown in FIG. 5. In FIG. 5(a) a residual plot is shown as a baseline where the residual is shown between the GBD values and a global average estimate. In FIG. 5(b), the residual is shown between the machine learning model prediction of FIG. 3 discussed above and the GBD values. The solid lines indicates the standard deviation. The plots show that the errors for diseases with a higher incidence rate are lower.

For the previously unseen countries application (FIG. 5), a decrease in the error magnitude at higher incidence rates was observed across the baseline and the trained network. This illustrates that both predictive models saw higher accuracy for common diseases whilst exhibiting a reduction in performance for rare diseases. However, this effect is more pronounced in the baseline model. It was observed that in the model trained with language embeddings, the error distribution was smaller with a shallower gradient illustrating the increase in performance.

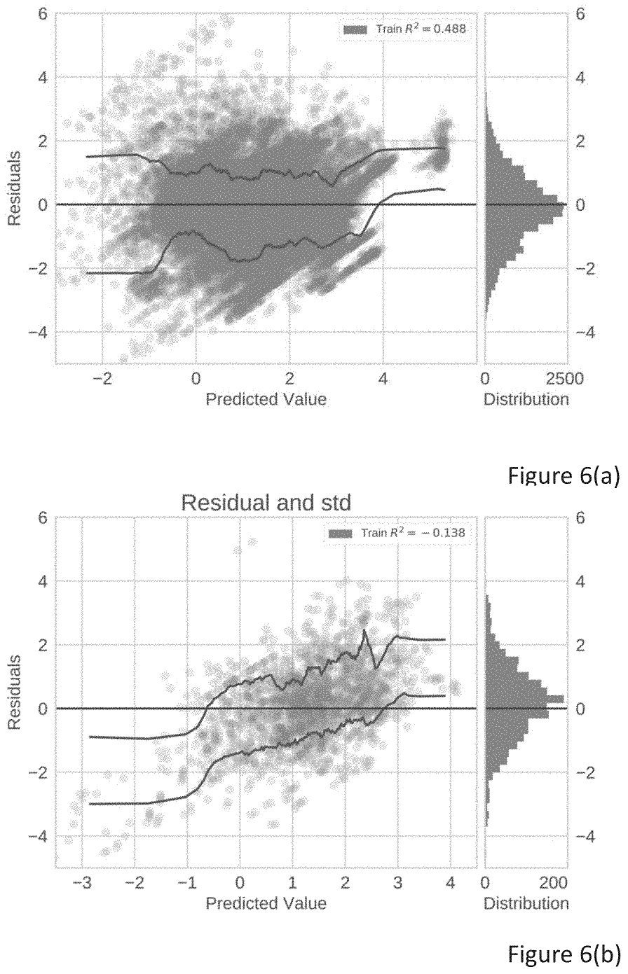

In the previously unseen diseases example, the error distribution in the GED validation set and the independent test set were analysed with data originating from peer-reviewed literature. This is shown in FIG. 6 where FIG. 6(a) shows the residual for the predicted value with respect to GBD data and FIG. 6(b) shows the residual for the predicted value with respect to the data from peer-reviewed journals. In the GBD validation set, the error distribution was constant with respect to the incidence magnitude. However, for the test-set, the model consistently over-predicted incidence rates especially at lower incidence diseases. It should be noted that the global average baseline comparison was not used here since a global average prediction is unrealistic in cases where the variance of incidence rates for a specific disease across countries is high. As above, the solid line shows the standard deviation.

In the above examples, the ability of different language models to encode contextual information was tested, and the corresponding embeddings were used as inputs to a neural network which was used to predict disease incidence. It was found that on average, models using BioBERT embeddings performed best across all metrics. High performance levels were observed when predicting for previously unseen countries and specific disease-country pairs, which was consistent across age groups.

Performance for previously unseen diseases was lower, varied substantially with age, and performance was notably lower for diseases which are highly dependent on location and climate. Overall, predictions were more accurate for common diseases than rare diseases.

BioBERT was the best-performing language model for creating disease embeddings for all three applications of their use; predicting disease incidence for previously unseen diseases, previously unseen countries, and specific disease-country pairs. The word embeddings for BioBERT were trained with text from medical journals and other clinical literature; this model should therefore have the most relevant context for interpreting words, which reflected in better disease incidence estimates from the neural network using these embeddings. Interestingly for previously unseen diseases and specific disease-country pairs, using feature fusion to combine information from the three language models resulted in substantially higher MAE than using BioBERT or other language models individually when the models were tested on external data from the GBD study. This suggests that using BioBERT alone results in sufficient contextual information, and further feature augmentation from other sources only adds redundant or correlated data.

When comparing the predictions to the GBD data, it was observed that performance for previously unseen diseases was significantly lower than for previously unseen countries and specific disease-country pairs. The purely data-driven neural network is able to predict disease incidence better for previously unseen countries and specific disease-country pairs because it already has data for the incidence of the disease it is trying to predict, and can draw sufficient context from the country embeddings to make a prediction for a new country. However, it is difficult to fully encapsulate how a previously unseen disease is similar to other diseases within a word embedding, and so the model's predictive ability is more limited for previously unseen diseases. This reflects the general state of knowledge; that it is possible to perform good inference for disease incidence in countries where data is lacking, based on knowledge of the country's socioeconomic situation, location, and healthcare provision, but struggle to predict the incidence of an unknown disease, regardless of how much data we have on other diseases in the same country. This is because the incidence of a disease is not only influenced by country-level factors but also by many biological, immunological, and sociodemographic factors.

Deep learning methods for predicting disease incidence, which use contextual embeddings learnt from unstructured information, have the potential to give better estimates of disease incidence than are currently available for settings where high quality data is lacking. This may be particularly valuable in areas lacking healthcare infrastructure where AI tools have the most potential to benefit people; settings where there is a lack of doctors, nurses, hospitals, etc. are very unlikely to have good data from which to estimate disease incidence.

In this work, we developed a machine learning method that is based on deep learning and transfer learning. The embedding methods were trained using a large amount of data while the target neural network for incidence estimation was only trained using data from the GBD study. As in many other data driven methods, the decision process of deep neural networks might overfit to the small training and validation dataset. Deep neural networks perform well on benchmark datasets, but can fail on real world samples outside the training data distribution. We have shown this effect by comparing the results on the validation and the test data. The performance on the test set that does not include examples from the training data was significantly lower.

Studies such as the GBD, which rigorously model disease statistics using information from multiple data sources, are limited by the time lag of data becoming available, and in their ability to incorporate new conditions due to the substantial effort involved in reviewing data and building new models. Whilst the above methods may be less rigorous, it is substantially quicker to implement for new diseases and can be easily updated to incorporate up-to-date contextual information for existing diseases, We therefore suggest it as a useful complement to existing modelling efforts, where data is required more rapidly or at larger scale than traditional methods allow for.

The above results show that the BioBERT language model performs well at encoding contextual information relating to disease incidence, and the resulting embeddings can be used as inputs to a neural network to successfully predict disease incidence for previously unseen countries and specific disease-country pairs, and for predicting for previously unseen diseases.

Further study was also performed into the nature of the embeddings. In an embodiment, it has been found that the word representations for either countries or diseases encapsulate relationships amongst each other. For instance, country embeddings for France and Spain can display similarities between each other that cover both geographical and socioeconomic metrics.

To evaluate the contextual meaning of the embeddings types, two classification experiments were performed where the input features are word embeddings obtained from either disease or country names and the labels to classify are either the GBD disease groups or country clusters. The resulting classification accuracy can serve as a metric to capture the contextual power of each embedding method when applied to either diseases or countries.

The first experiment aimed at evaluating whether disease embeddings capture context and similarities between diseases. In an embodiment, the input features are word embeddings obtained from disease names and the labels to predict are the 17 high-level GBD disease groups:

GBD disease groups
HIV/AIDS and sexually transmitted infections: Genital herpes, Trichomoniasis, Syphilis, Chlamydial infection, HIV/AIDS, Gonococcal infection
Respiratory infections and tuberculosis: Lower respiratory infections, Tuberculosis, Upper respiratory infections, Otitis media
Enteric infections: Invasive Non-typhoidal Salmonella (iNTS), Diarrheal diseases, Typhoid fever, Paratyphoid fever
Neglected tropical diseases and malaria: Malaria, Leprosy, Dengue, Visceral leishmaniasis, Cutaneous and mucocutaneous leishmaniasis, African trypanosomiasis, Rabies, Zika virus, Food-borne trematodiases, Cystic echinococcosis, Chagas disease, Ebola, Guinea worm disease, Yellow fever
Other infectious diseases: Encephalitis, Diphtheria, Measles, Tetanus, Varicella and herpes zoster, Acute hepatitis C, Meningitis, Acute hepatitis B, Acute hepatitis A, Whooping cough, Acute hepatitis F
Nutritional deficiencies: Iodine deficiency, Protein-energy malnutrition, Vitamin A deficiency
Neoplasms: Lip and oral cavity cancer, Esophageal cancer, Acute myeloid leukemia, Brain and nervous system cancer, Nasopharynx cancer, Acute lymphoid leukemia, Mesothelioma, Kidney cancer, Non-Hodgkin lymphoma, Myelodysplastic, myeloproliferative, and other hematopoietic neoplasms, Non-melanoma skin cancer (basal-cell carcinoma), Breast cancer, Testicular cancer, Bladder cancer, Chronic lymphoid leukemia, Stomach cancer, Thyroid cancer, Larynx cancer, Multiple myeloma, Liver cancer, Tracheal, bronchus, and lung cancer, Colon and rectum cancer, Other pharynx cancer, Pancreatic cancer, Chronic myeloid leukemia, Hodgkin lymphoma, Prostate cancer, Benign and in situ intestinal neoplasms, Non-melanoma skin cancer (squamous-cell carcinoma), Gallbladder and biliary tract cancer, Malignant skin melanoma
Cardiovascular diseases: Intracerebral hemorrhage, Peripheral artery disease, Endocarditis, Subarachnoid hemorrhage, Non-rheumatic calcific aortic valve disease, Non-rheumatic degenerative mitral valve disease, Myocarditis, Atrial fibrillation and flutter, Ischemic heart disease, ischemic stroke, Rheumatic heart disease
Chronic respiratory diseases: Chronic obstructive pulmonary disease, Interstitial lung disease and pulmonary sarcoidosis, Asbestosis, Asthma, Silicosis, Coal workers pneumoconiosis
Digestive diseases: Gallbladder and binary diseases, Appendicitis, Cirrhosis and other chronic liver diseases, Peptic ulcer disease, Inguinal, femoral, and abdominal hernia, Gastritis and duodenitis, Inflammatory bowel disease, Pancreatitis, Paralytic ileus and intestinal obstruction, Vascular intestinal disorders, Gastroesophageal reflux disease
Neurological disorders: Multiple sclerosis, Parkinson's disease, Epilepsy, Motor neuron disease, Alzheimer's disease and other dementias, Migraine, Tension-type headache
Mental disorders: Conduct disorder, Schizophrenia, Major depressive disorder, Dysthymia, Bulimia nervosa, Bipolar disorder, Anxiety disorders, Attention-deficit/hyperactivity disorder, Anorexia nervosa
Substance use disorders: Alcohol use disorders, Cannabis use disorders, Opioid use disorders, Cocaine use disorders, Amphetamine use disorders
Diabetes and kidney diseases: Diabetes mellitus type 2, Acute glomerulonephritis, Diabetes mellitus type 1, Chronic kidney disease
Skin and subcutaneous diseases: Acne vulgaris, Pruritus, Contact dermatitis, Atopic dermatitis, Viral skin diseases, Urticaria, Decubitus ulcer, Pyoderma, Fungal skin diseases, Alopecia areata, Cellulitis, Seborrhoeic dermatitis, Psoriasis, Scabies
Musculoskeletal disorders: Gout, Rheumatoid arthritis, Low back pain, Neck pain, Osteoarthritis
Other non-communicable diseases: Benign prostatic hyperplasia, Periodontal diseases, Urolithiasis, Edentulism and severe tooth loss, Urinary tract infections, Caries of deciduous teeth, Caries of permanent teeth

The embeddings computed from countries and can capture both geographical and economic dimensions. This can be evaluated by considering the classification of 21 country clusters such as “High-Income Asia Pacific” and “Western Europe” from country embeddings.

### Country Clusters

North Africa and Middle East: Lebanon, Libya, Morocco, Oman, Syria, Tunisia, Palestine, Turkey, United Arab Emirates, Egypt, Algeria, Yemen, Iran, Afghanistan, Qatar, Kuwait, Bahrain, Jordan, Iraq, Saudi Arabia, Sudan
South Asia: Bhutan, Pakistan, India, Nepal, Bangladesh
Central Asia: Azerbaijan, Georgia, Armenia, Kazakhstan, Tajikistan, Uzbekistan, Kyrgyzstan, Mongolia, Turkmenistan
Central Europe: Bosnia and Herzegovina, Czech Republic, Bulgaria, Croatia, Hungary, Montenegro, Romania, Serbia, Macedonia, Poland, Slovenia, Slovakia, Albania
Eastern Europe: Belarus, Latvia, Lithuania, Moldova, Russian Federation, Ukraine, Estonia
Australasia: Australia, New Zealand
High-income Asia Pacific: Brunei, Japan, Singapore, South Korea
High-income North America: Canada, United States, Greenland
Southern Latin America: Argentina, Chile, Uruguay
Western Europe: Italy, Malta, Andorra, Netherlands, Israel, United Kingdom, Norway, Portugal, Cyprus, Switzerland, Spain, Sweden, Ireland, Luxembourg, Denmark, Greece, Austria, Belgium, Finland, Germany, Iceland, France
Andean Latin America: Bolivia, Peru, Ecuador
Caribbean: Antigua and Barbuda, Puerto Rico, The Bahamas, Dominican Republic, Barbados, Belize, Dominica, Virgin Islands, U.S., Grenada, Guyana, Haiti, Cuba, Suriname, Saint Lucia, Saint Vincent and the Grenadines, Trinidad and Tobago, Jamaica, Bermuda
Central Latin America: Colombia, Costa Rica, El Salvador, Honduras, Mexico, Guatemala, Nicaragua, Panama, Venezuela
Tropical Latin America: Brazil, Paraguay
East Asia: China, North Korea, Taiwan
Oceania: Kiribati, Marshall Islands, Fiji, Northern Mariana islands, Federated States of Micronesia, Papua New Guinea, Solomon Islands, Samoa, Tonga, Vanuatu, American Samoa, Guam
Southeast Asia: Cambodia, Laos, Philippines, Maldives, Indonesia, Myanmar, Vietnam, Malaysia, Sri Lanka, Timor-Leste, Thailand, Seychelles, Mauritius
Central Sub-Saharan Africa: Angola, Central African Republic, Congo, Democratic Republic of the Congo, Equatorial Guinea, Gabon
Eastern Sub-Saharan Africa: Somalia, Djibouti, Uganda, Tanzania, Burundi, Comoros, Madagascar, Ethiopia, Eritrea, Rwanda, South Sudan, Zambia, Kenya, Mozambique, Malawi
Southern Sub-Saharan Africa: Botswana, South Africa, Swaziland, Lesotho, Zimbabwe, Namibia
Western Sub-Saharan Africa: Guinea-Bissau, Liberia, Mauritania, Mali, Niger, Sierra Leone, Togo, Guinea, Senegal, Sao Tome and Principe, Nigeria, Benin, Burkina Faso, Cameroon, Chad, Cape Verde, “Cote dIvoire”, The Gambia, Ghana

Linear Support Vector Machines were trained for each classification experiment across a candidate set of model hyperparameters. Models were trained and evaluated using 3-fold cross-validation. The cross-validation experiments were repeated 10 times to mitigate any potential bias in the training and validation split. The best performing models for each embedding across both experiments were then used to assess the accuracy.

Results for the classification experiments are shown below in Table 4.

TABLE 4
Classification results for GBD disease groups using disease embeddings
and country clusters with country embeddings. Reported results
are from 10-repeated 3-fold cross-validation experiments.

| | GBD disease groups | | | Country dusters | | |
|---|---|---|---|---|---|---|
| model | GloVe | BioBERT | USE | GloVe | BioBERT | USE |
| accuracy | 0.77 (0.03) | 0.77 (0.02) | 0.66 (0.02) | 0.73 (0.02) | 0.17 (0.02) | 0.62 (0.03) |

The average and standard-deviation of the model accuracy across cross-validation folds are reported. For the GBD disease group classification, equitable performance was observed across the GloVe and BioBERT disease embeddings with 0.77 accuracy whilst USE embeddings saw 0.66 accuracy.

For the country cluster classification, the highest performance was observed across GloVe embeddings with 0.73 compared to 0.17 and 0.62 for BioBERT and USE respectively. This illustrates how GloVe country embeddings capture meaningful relationships between countries whilst BioBERT country embeddings are ineffective as they were trained on large-scale biomedical corpora not useful for countries.

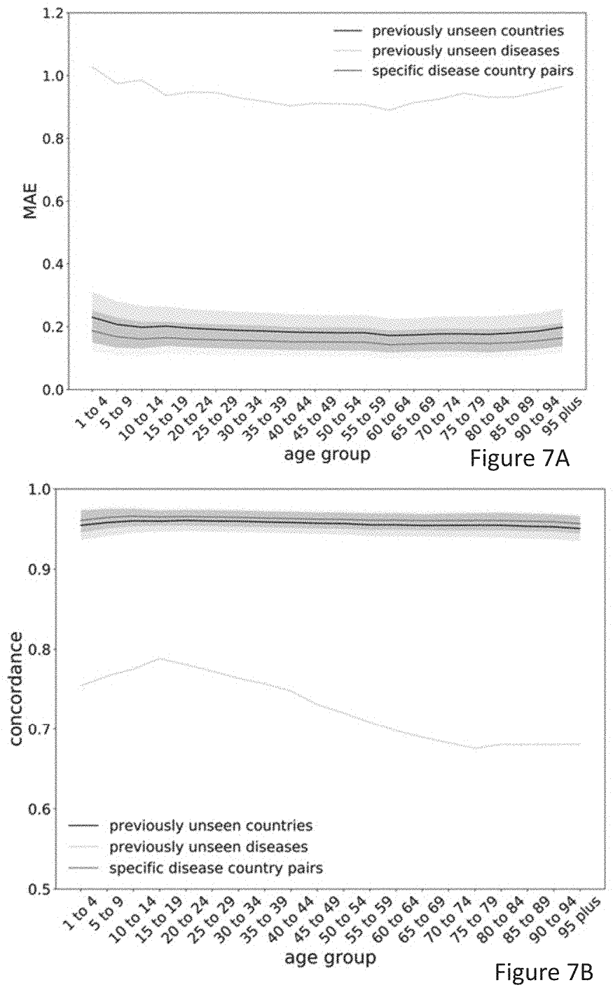

The performance of the BioBERT model across all applications stratified by age-group was studied. Both the concordance (FIG. 7(b)) and MAE (FIG. 7(a)) were quantified between predicted incidence rates and the ground truth. Across the previously unseen countries application (dark grey) and the specific disease-country pairs application (mid grey), the performance was consistently high and constant across all age-groups. In contrast, in the previously unseen diseases (light grey) application that aimed at predicting unknown target diseases across all countries, the MAE and the concordance varied with age-group with best performance (high concordance, low MAE) across adults and sharp drops at both extremes of the age spectrum.

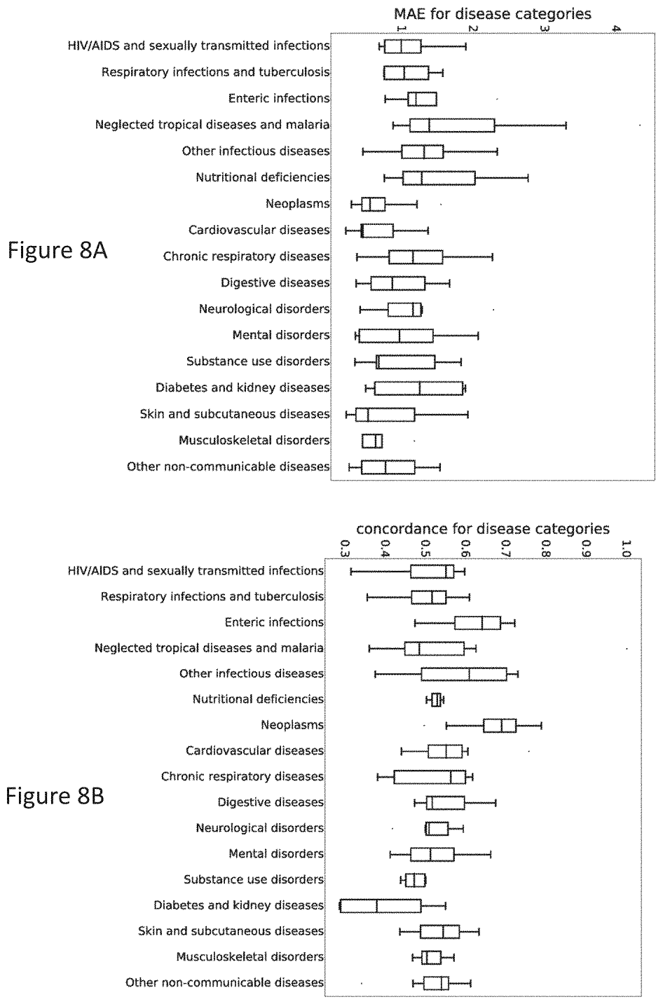

The performance was analysed for the previously unseen diseases by evaluating the model across 17 disease groups based on the GBD model of diseases. The MAE (FIG. 8(a)) and concordance (FIG. 8(b)) were calculated between predicted values and the ground-truth with the standard-deviation of these measures computed over the cross-validation folds. The three disease groups with the highest error were: 1) neglected tropical diseases and malaria, 2) Other infectious disease and 3) nutritional deficiencies. Diseases stemming from these groups are generally difficult to predict accurately since they are highly dependent on location and climate.

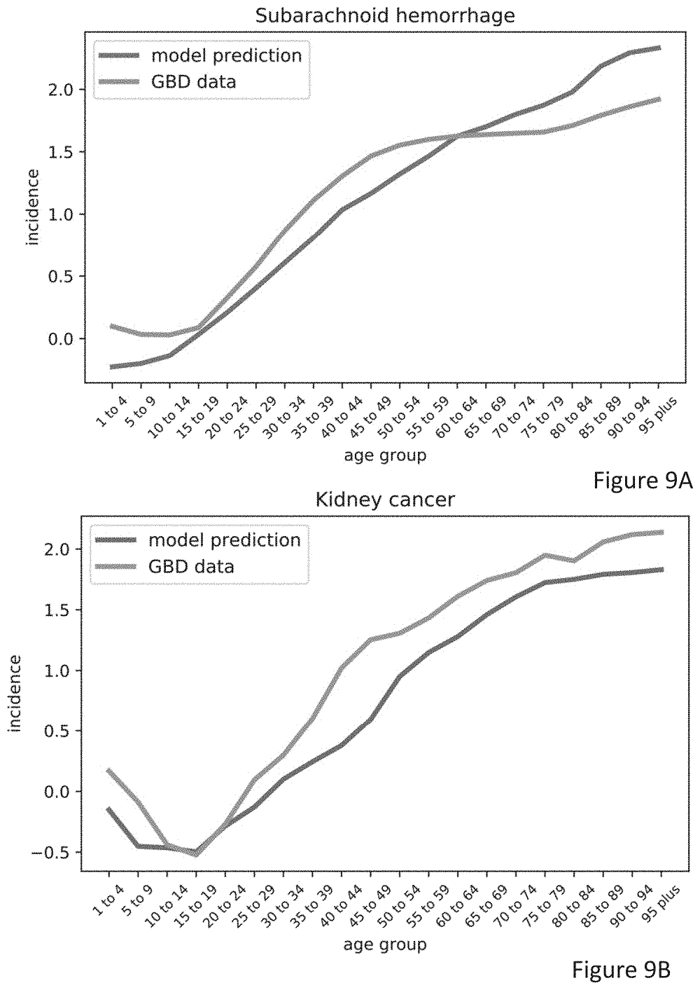

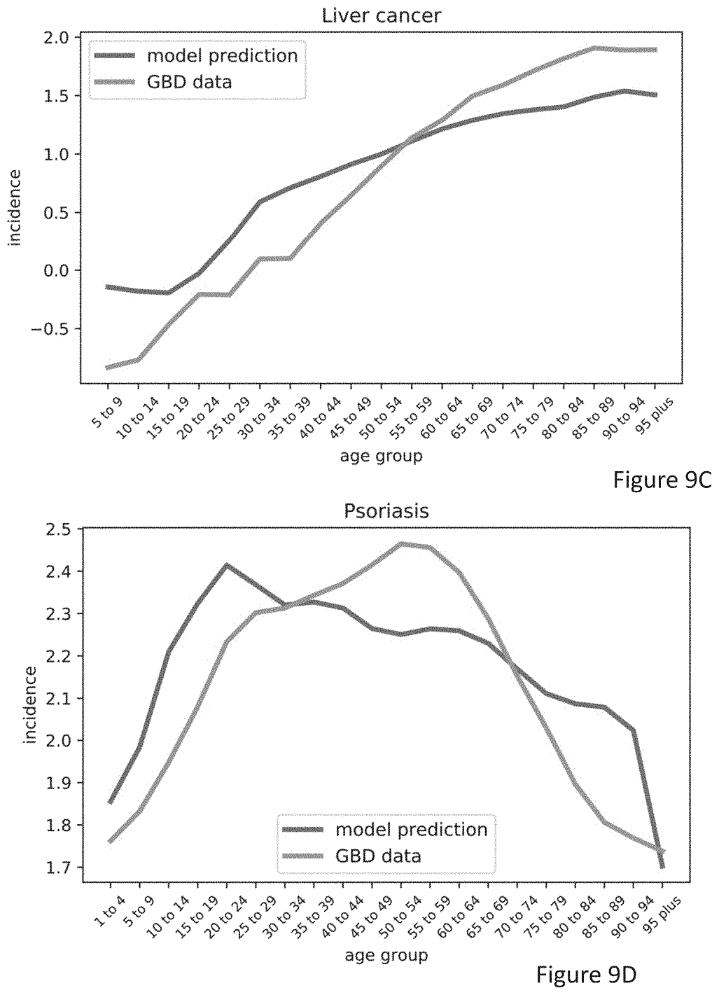

A further experiment was also performed where incidence rates for new diseases within the United Kingdom were estimated, which falls under the previously unseen diseases application. FIG. 9 illustrates the four diseases for which the model made the most accurate predictions; a) subarachnoid hemorrhage, b) kidney cancer, c) liver cancer and d) psoriasis.

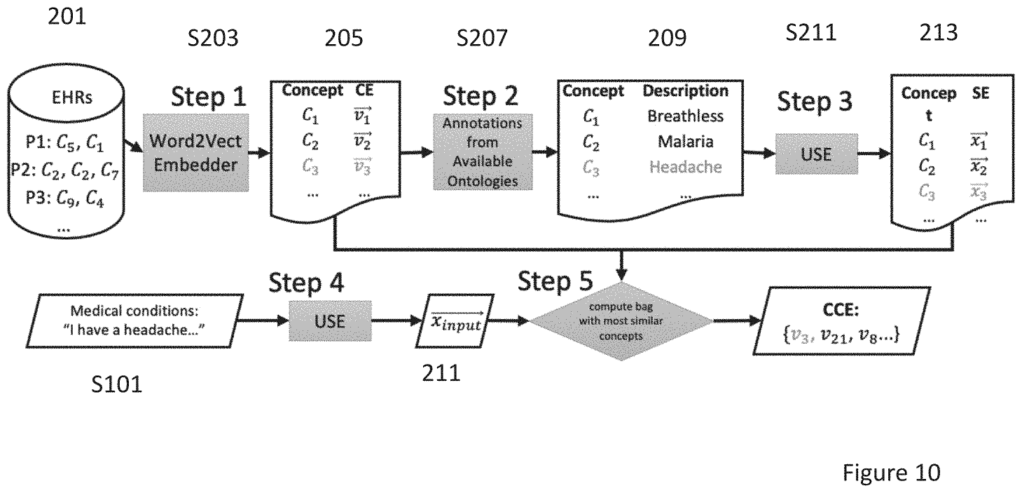

A further type of embedding will now be described with reference to FIGS. 10 to 15,

FIG. 10 is a flow diagram showing the overall principles.

In FIG. 10, database 201 is provided. The data based comprises a plurality of clinical records with a record for each patient: P1, P2, P3 . . . etc, Each patient record, P1 etc, comprises a plurality of medical concepts: C1, C2, . . . etc.

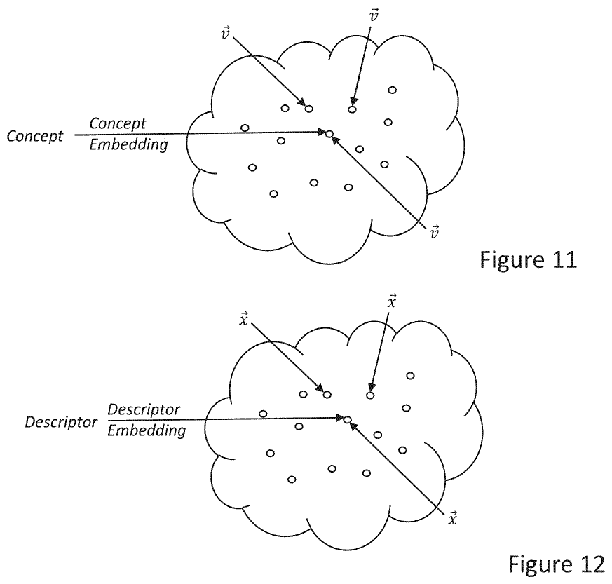

In an embodiment, these concepts are then used to train an embedder such that an embedder in step S203 can produce an embedded concept vector \vec{vᵢ} corresponding to each concept i. For example, the embedder may be trained using skipgram. FIG. 11 is a schematic showing the embedded space for a concept vector.

In further embodiments a pre-trained embedder is used. The aim of this step is to provide an embedded space for clinical concepts as shown in FIG. 11. The space allows similar concepts to be identified, or concepts that occur together.

The output of the embedder is a dictionary of clinical concepts Cᵢ and their corresponding embedded vectors \vec{vᵢ} as shown in 205 Each clinical concept will also have a corresponding descriptor which is available from known medical ontologies, for example, SNOMED. The descriptor will provide text related to the concept. For example:

ConceptID 22298006:
Fully specified name; Myocardial infarction (disorder)
Preferred term: Myocardial infarction
Synonym: Cardiac infarction
Synonym: Heart Attack
Synonym: Infarction of heart

The descriptor or descriptors are retrieved in step S207 to provide library 209. The descriptors from library 209 are then put through an embedder, for example, a universal sentence embedder (USE) in step S211 to produce an embedded sentence output library 213 which contains concepts Ci and their corresponding embedded vector.

FIG. 12 shows schematically the descriptor embedded space that is different to the concept embedding space of FIG. 11.

The above results in dictionaries 205 and 213, where dictionary 205 links concepts Ci with their embedded representations \vec{vᵢ} established on the basis of the occurrence of these concepts happening and library 213 that links concepts Ci with an embedded vector \vec{xᵢ} based on their descriptors.

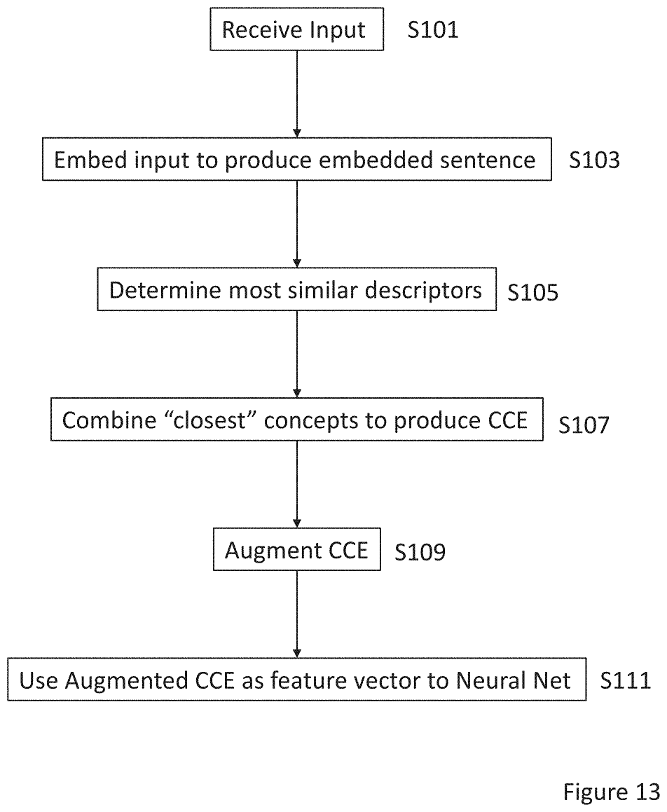

This completes the training of a model that is then used to produce a clinical context embedding (CCE) that will be described with reference to FIG. 13 as well as FIG. 10.

In step S101 of FIGS. 10 and 13, an input is received. This input can be, for example, any clinical term. However, for this example, it will be presumed that it is a disease. In step S103, the text input is then embedded into the first embedding space, using the same linguistic embedder that was discussed in S211 in FIG. 10.

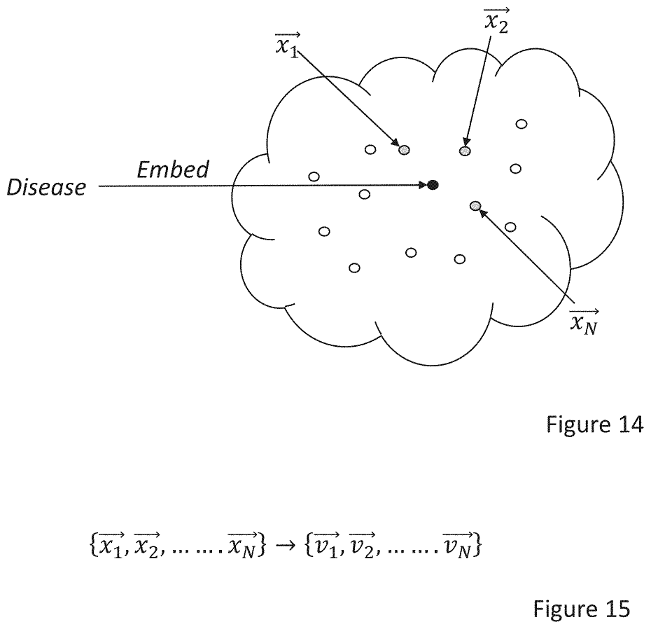

In step S105, the n closest first embedded vectors are determined as shown in FIG. 14. In an embodiment, the Euclidean distance as a similarity metric. Here, n is a hyperparameter which can be optimised on a validation set.

Once the n closest first embedded vectors are determined, these are mapped to their corresponding concept vectors. The corresponding concept vectors were determined in step S203 of FIG. 12. The correspondence between the first embedded vectors and their corresponding concept vectors can be determined offline. This can then be saved in a database to access during run-time as shown in FIG. 15.

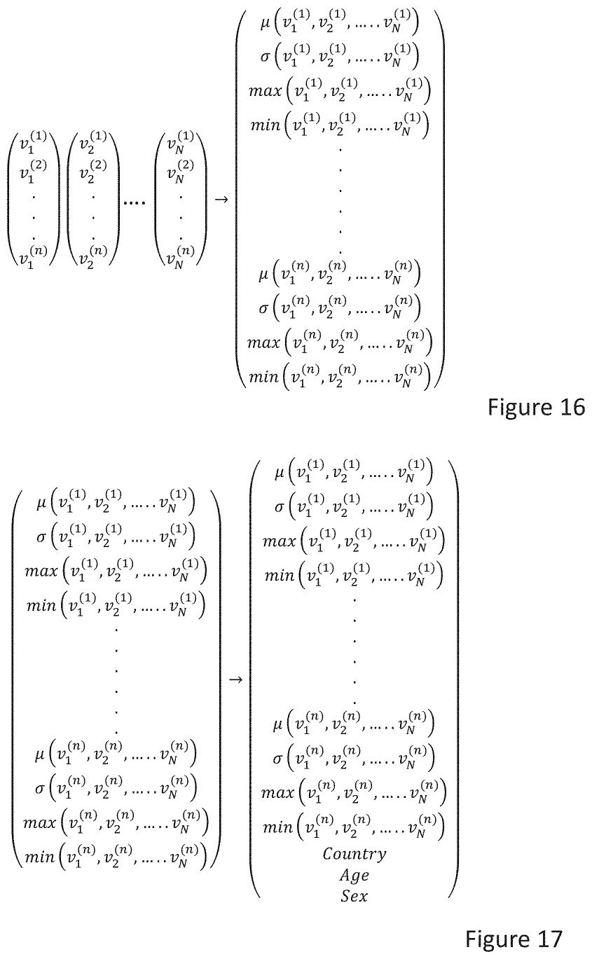

In step S107, these most similar concepts are then combined to produce a context vector. FIG. 16 shows how the concepts vectors are combined, To derive a size-independent vectorized representation of the bag of concept vectors, in the embodiment, the mean, standard deviation, minimum and maximum values across the n concept vectors in the bag are concatenated and these values are combined to form a resulting context vector, that will be called the Contextual Clinical Embedding (CCE). Here, the size of the CCEs is 4 times the size of the concept vectors and is independent of the number of concept vectors n.

The use of the CCE allows the handling of out-of-vocabulary cases. The Contextual Clinical Embedding (CCE) is a representation of concepts that are contextually most similar to any text input.

Once computed, the CCEs can be used for different ML tasks such as clustering, classification or regression.

The embodiments described herein deal with out-of-vocabulary (OOV) cases. To do this, the embodiment utilises the Universal Sentence Encoder (USE) to search for CEs with high semantic similarity. This allows the embodiment to compute a vectorised representation of free text that can denote or describe a disease and was not in the vocabulary of the training set.

In a further embodiment, in an embodiment, the representations are used, together with country, age and gender embeddings as shown in FIG. 17.

The above examples have described the method for predicting the prevalence or incidence of a condition where there is no data for the condition within a country or even where possibly the condition is unknown. However, it is possible to use the method to also predict the conditional probability between diseases and symptoms and this in turn allows for links to be added to the PGM.

The basic method in shown in FIG. 18, Block 1455 represents the language model that converts a condition 1451 into embedded disease vector 1457 and a symptom 1453 into embedded symptom vector 1458. The embedded condition vector 1457 and embedded symptom vector are then concatenated together to form a single embedded vector, this is then input into trained neural network 1459 to output a conditional probability on the conditions and symptoms 1461.

Trained neural network 1459 is trained on known marginals for disease and symptom pairs.

Due to the use of the language model, similarities between symptoms is leveraged and similarities between conditions is leveraged. Thus, once the model is trained, a symptom and disease where their relation is unknown can be embedded through embedder 1455 to produce an embedded disease vector and an embedded symptom vector which are then concatenated to be input into the trained model. In an embodiment, the known marginals were provided as binary labels (i.e. the presence or absence of a link), for example, links with a high marginal probability were chosen to indicate the presence of a link.

In an embodiment, the probability P(D, S) is calculated with a softmax function, but other functions could be used. The above can be used for a disease and symptom pair where possibly the symptom and/or the disease is unknown to the model since the embedding allows the system to understand and leverage similarities between symptoms and leverage similarities between symptoms.

The above can therefore be used to enhance the PGM of FIG. 2 by providing further information. It is also possible for the above method to be used to predict new links in a PGM. FIG. 19 is a schematic of a PGM with diseases “D” and Symptoms “S”. with conditions D₁, D₂ and D₃ and Symptoms S₁, S₂ and S₃. It is desired to introduce a new symptom S₄ into the PGM, but it is not known how this symptom is linked to the various conditions.

To build these links into the PGM, it is possible to produce an embedded disease vector for disease D₃ and an embedded symptom vector S₄. These vectors then concatenated and provided to network 1459. The output from network 1459 can then be compared with a threshold. For example, 0.5. If the output (D, S) is above the threshold, then it is determined that a link is present this is added to the PGM. On the other hand, if the output is below the threshold, then no link is determined. In FIG. 19, it is tested whether there are links between C₂ and S₄ and C₃ and S₄.

In the above description, the disease and the symptom is embedded using the same language model. However, this is not necessary. It is possible for them to be embedded using the same or different language models. Also, as described above, it is possible for there to be a combination of different language models which are combined. It is also possible for the embedding described with reference to FIGS. 10 to 17 can also be used which uses a language model which is enhanced with concept information.

The value output from network 1461 is dependent on the training data. To determine the presence or absence of links, the network can be trained on binary labels as described above. In further embodiments, the network 1461 can be trained on P(S,D) data or P(S|D) where exact values of known conditionals are used. In practice, it is difficult to obtain correct values of conditionals to produce a large training set.

As an example, in a network trained on condition pairs, the following links were suggested where Headache was provided as a condition:

Headache as symptom->suggested link to disease

1. Intermittent headache

->Mumps

->Bacterial tonsillitis

->Subdural haemorrhage

->Chalazion

->Otitis media

->Subarachnoid haemorrhage

->Acoustic neuroma

->Hemorrhagic cerebral infarction

->Lyme disease

->Viral meningitis

->Optic neuritis

2. Unilateral headache

->Analgesia overuse headache

->Ischemic stroke

->Bruxism

->Benign intracranial hypertension

->Viral meningitis

->influenza

->Infectious mononucleosis

->Hypoglycaemic episode

->Premenstrual tension syndrome

3. Occipital headache

->Strain of neck muscle

->Trigeminal neuralgia

->Analgesia overuse headache

->Temporal arteritis

->Scleritis

->Hemorrhagic cerebral infarction

->Pituitary adenoma

->Cluster headache

4. Temporal headache

->Secondary hypertension

->Acute sinusitis

->Otitis media

->Hypoglycaemic episode

->Malignant neoplasm of brain

->Viral meningitis

### Headache as Disease->Suggested Link to Symptom

1. Cluster headache

->Photopsia (unilateral)

->Unilateral arm numbness

->Occipital headache

2. Analgesia overuse headache

->Unilateral headache

->Occipital headache

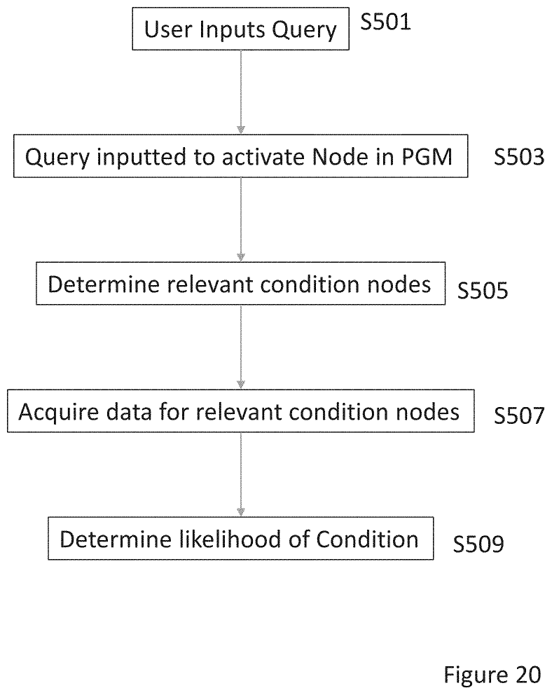

FIG. 20 is a flow diagram showing how the data predicted above can be used in the system of FIG. 1.

The user inputs a query via their mobile phone, tablet, PC or other device at step S501. The user may be a medical professional using the system to support their own knowledge or the user can be a person with no specialist medical knowledge. The query can be input via a text interface, voice interface, graphical user interface etc. In this example, it will be assumed that the query is a user inputting a symptom.

As explained with reference to FIGS. 1 and 2, the query is processed by the interface such that a node in the PGM that corresponds to the query can be recognised (or “activated”) in step S503.

Once a node in the PGM is activated, it is possible to determine the relevant condition nodes (i.e. the nodes which corresponds to conditions that are linked to the activated node) in step S505.

The system will be aware of various characteristics such as the country where the user is located, their age, gender etc. These characteristics might be held in memory for a user or they may be requested each time from a user.

In step S507, the marginals are determined for each of the relevant condition nodes. As explained above in relation to FIG. 2, the aim is to determine the likelihood of a disease given that a symptom is present. As shown in equation 1 above, it is necessary to determine the prevalence of the disease P(D) and the marginal P(S|D) both of are determined from the PGM. Some of the values of the PGM will be determined from studies. However, the above described methods allow the PGM to be populated with further P(D) values to allow diseases to be considered that would otherwise not be able to be considered due to the data not being available. Also, the above methods allow extra links in the PGM to be provided if the method described with reference to FIGS. 18 and 19 has been used to enhance the PGM.

The likelihood of a disease being present is then determined using inference on the PGM and the marginals and prevalence.

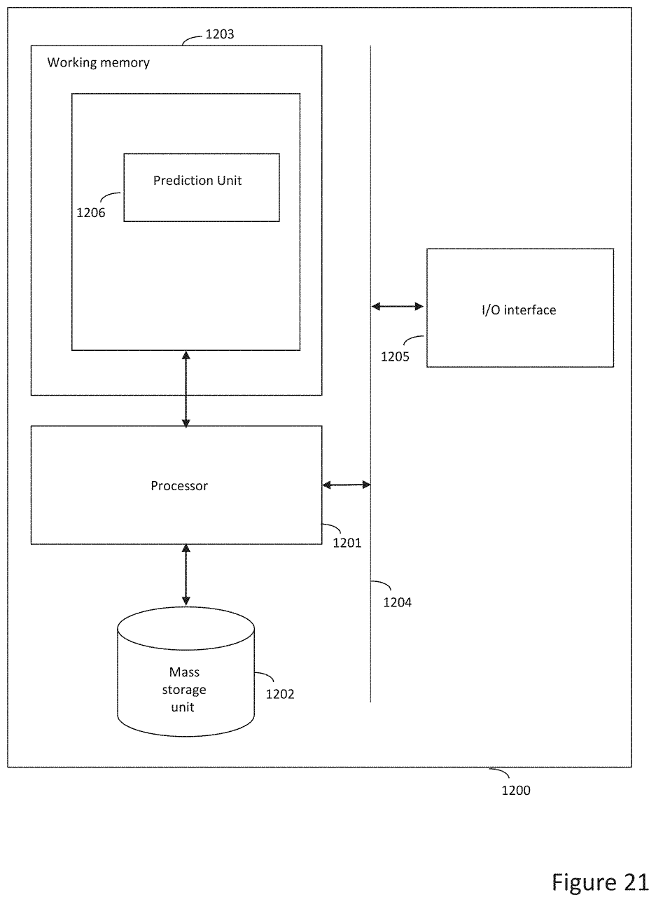

While it will be appreciated that the above embodiments are applicable to any computing system, an example computing system is illustrated in FIG. 21, which provides means capable of putting an embodiment, as described herein, into effect. As illustrated, the computing system 1200 comprises a processor 1201 coupled to a mass storage unit 1203 and accessing a working memory 1203. As illustrated, a prediction unit 1206 is represented as software products stored in working memory 1203. However, it will be appreciated that elements of the prediction unit 1206, may, for convenience, be stored in the mass storage unit 1202.

Usual procedures for the loading of software into memory and the storage of data in the mass storage unit 1202 apply. The processor 1201 also accesses, via bus 1204, an input/output interface 1205 that is configured to receive data from and output data to an external system (e.g. an external network or a user input or output device). The input/output interface 1205 may be a single component or may be divided into a separate input interface and a separate output interface.

Thus, execution of the prediction unit 1206 by the processor 1201 will cause embodiments as described herein to be implemented.

The, a prediction unit 1206 can be embedded in original equipment, or can be provided, as a whole or in part, after manufacture. For instance, the prediction unit 1206 can be introduced, as a whole, as a computer program product, which may be in the form of a download, or to be introduced via a computer program storage medium, such as an optical disk. Alternatively, modifications to existing prediction unit 1206 software can be made by an update, or plug-in, to provide features of the above described embodiment

The computing system 1200 may be an end-user system that receives inputs from a user (e.g. via a keyboard) and retrieves a response to a query using prediction unit 1206 adapted to produce the user query in a suitable form. Alternatively, the system may be a server that receives input over a network and determines a response. Either way, the use of the prediction unit 1206 may be used to determine appropriate responses to user queries, as discussed with regard to FIG. 1.

Implementations of the subject matter and the operations described in this specification can be realized in digital electronic circuitry, or in computer software, firmware, or hardware, including the structures disclosed in this specification and their structural equivalents, or in combinations of one or more of them. Implementations of the subject matter described in this specification can be realized using one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices)

While certain embodiments have been described, these embodiments have been presented by way of example only, and are not intended to limit the scope of the inventions. Indeed the novel methods and systems described herein may be embodied in a variety of other forms; furthermore, various omissions, substitutions and changes in the form of methods and systems described herein may be made without departing from the spirit of the inventions. The accompanying claims and their equivalents are intended to cover such forms of modifications as would fall within the scope and spirit of the inventions.

1. A computer implemented method for developing a probabilistic graphical representation, the probabilistic graphical representation comprising nodes and links between the nodes indicating a relationship between the nodes, wherein the nodes represent conditions, the method comprising:
using a language model to produce a context aware embedding for said condition;
enhancing said embedding with one or more features to produce an enhanced embedded vector; and
using a machine learning model to map said enhanced embedded vector to a value, wherein said value is related to the node representing said condition or a neighbouring node,
wherein said machine learning model has been trained using said enhanced embedded vectors and observed values corresponding to said enhanced embedded vectors.

2. A computer implemented method according to claim 1, wherein enhancing the embedding with one or more features comprises:
enhancing said embedding with one or more labels, said labels providing information concerning a population, wherein the value represents the prevalence or incidence of the condition in the population and adding said value to the node corresponding to the condition in the probabilistic graphical representation.

3. A computer implemented method according to claim 2, wherein the labels are one or more selected from location, age and sex of population.

4. A computer implemented method according to claim 1, wherein enhancing the embedding with one or more features comprises:
using a language model to produce a context aware embedding for a further condition and concatenating the embedding for the further condition with that of the embedding for the said condition to produce an enhanced embedded vector,
wherein the value represents a probability that the two conditions occur together.

5. A computer implemented method according to claim 4,
the method further comprising comparing the said value with a threshold, the method determining the presence of a link in the probabilistic graphical model between the two conditions if the value is above the threshold and adding the link to the probabilistic graphical representation.

6. A computer implemented method according to claim 2, wherein location is expressed as an embedded vector.

7. A computer implemented method according to claim 1, wherein the language model is adapted to receive free text.

8. A computer implemented method according to claim 1, wherein the language model has been trained on a biomedical database.

9. A computer implemented method according to claim 1, wherein said language model is selected from BioBERT, GloVe, USE or GPT.

10. A computer implemented method according to claim 1, wherein a plurality of said embeddings are used to produce said context aware embedding.

11. A computer implemented method according to claim 2, wherein said label is location and the method is used to determine a value for a condition in a specified location and the machine learning model has been trained on values for conditions including this condition for other locations and for other conditions for the specified location.

12. A computer implemented method according to claim 2, wherein said label is location and the method is used to determine a value for a condition in a specified location and the machine learning model has been trained on values for conditions not including this condition for any location.

13. A computer implemented method according to claim 2, wherein said label is location and the method is used to determine a value for a condition in a specified location and the machine learning model has been trained on values for conditions, but not on any values concerning the specified location.

14. A computer implemented method according to claim 4, wherein the embedded vector comprises embedded vectors from two conditions the method is used to determine a value of the probability of both conditions occurring and the machine learning model has been trained on values that do not include the two conditions that form the embedded vector and an input.

15. A computer implemented method according to claim 1, wherein using a language model to produce a context aware embedding for said condition comprises:
embedding input text into a first embedded space, wherein said first embedded space comprises first vector representations of descriptors of concepts in a knowledge base;
selecting the n nearest neighbours said first embedded space, wherein the nearest neighbours are first vector representations of descriptors and n is an integer of at least 2;
acquiring second concept vector representations of the concepts corresponding to the descriptors of said n nearest neighbours, wherein the second concept vector representations of said concepts is based on relations between said concepts; and
combining said second concept vector representations into a single vector to produce said context vector representation of said input text.

16. A computer implemented method according to claim 11, wherein combining said second concept vector representations comprises:
for each dimension of said concept vector representations, obtaining a statistical representation of the values for the same dimension across the selected concept vector representations to produce a dimension in said context vector representation of said input text.

17. A computer implemented method according to claim 12, wherein the statistical representations are selected from: mean, standard deviation, min and max.

18. A method of determining the likelihood of a user having a condition, the method comprising:
inputting a symptom into an inference engine, wherein said inference engine is adapted to perform probabilistic inference over a probabilistic graphical representation,
said probabilistic graphical representation comprising diseases and symptoms and the probabilities linking these diseases and symptoms, and wherein at least one of the probabilities is determined from the values derived according to the method of claim 1.

19. A system for developing a probabilistic graphical representation, the probabilistic graphical representation comprising nodes and links between the nodes indicating a relationship between the nodes, wherein the nodes comprise nodes representing conditions and nodes representing symptoms, the system comprising:
a processor adapted to:
use a language model to produce a context aware embedding for said condition;
enhance said embedding with one or more features to produce an enhanced embedded vector; and
retrieve from memory a machine learning model, said machine learning model being configure to map said enhanced embedded vector to a value, wherein said value is related to the node representing said condition or a neighbouring node,
wherein said machine learning model has been trained using said enhanced embedded vectors and observed values corresponding to said enhanced embedded vectors.

20. A non-transitory computer-readable medium comprising instructions which, when executed by a computer, cause the computer to carry out the method of claim 1.
